(19) United States
(12) Patent Application Publication
(10) Pub. No.: US 2026/0207446 A1
(43) Pub. Date: Jul. 23, 2026

(54) ADDUCTS OF POLYAMINO ACIDS, ALKALI METAL PYROPHOSPHATES AND PEROXIDES

(71) Applicant: ISP Investments LLC, Wilmington, DE (US)

(72) Inventors: Petros GEBRESELASSIE, Whitehouse Station, NJ (US); Thomas WINKLER, Maywood, NJ (US); Philip John Oths, Mendham, NJ (US); Sounak SARKAR, Hillsborough, NJ (US); Hani M. FARES, Sommerset, NJ (US)

(73) Assignee: ISP Investments LLC, Wilmington, DE (US)

(21) Appl. No.: 19/136,074

(22) PCT Filed: Dec. 5, 2023
(86) PCT No.: PCT/US2023/082542

§ 371 Date: Jun. 5, 2025

Related U.S. Application Data
(60) Provisional application No. 63/430,155, filed on Dec. 5, 2022.

(51) Int. Cl. A61K 8/22; A61K 8/24; A61K 8/64; A61K 8/88; A61Q 11/00

(52) U.S. Cl. CPC A61K 8/22; A61K 8/24; A61K 8/64; A61K 8/88; A61Q 11/00

(57) ABSTRACT

Disclosed is an adduct of (i) peroxide; (ii) alkali metal pyrophosphates, a stabilizing agent; and (iii) at least one compound selected from the group consisting of polyamino acids, proteins, and any combinations thereof. Also disclosed a composition(s) comprising the adduct of the present disclosure, particularly an oral care composition.

### FIELD OF THE INVENTION

The presently disclosed process(es), procedure(s), method(s), product(s), result(s), and/or concept(s) (collectively referred to hereinafter as the “present disclosure”) relates generally to adduct of peroxides, polyamino acids including their salts and alkali metal pyrophosphates. The present disclosure also relates to a process(s) for preparing the same and compositions derived from the adducts of the present disclosure, particularly oral care compositions.

### BACKGROUND OF THE INVENTION

The increasing demand for a whiter smile has made teeth bleaching a popular means of obtaining whiter teeth. Over the counter (OTC) bleaching products for unsupervised home applications are commonly used to whiten teeth. Non-bleaching toothpastes are only effective at removing extrinsic stains, whereas teeth bleaching products remove intrinsic stains that are deposited deep in the teeth structure. Teeth bleaching practices have expanded rapidly from one in which a person could only bleach the teeth in a dental office to the current practice where several OTC teeth bleaching products are available for anyone to use unsupervised at home. Although bleaching treatments administrated by a dentist achieves significantly better whitening results, self-administrated bleaching products have been shown to provide a noticeable whitening effect within a few days of use at significantly lower cost. Hydrogen peroxide (H₂O₂) is the most preferred and commonly used bleaching agent in OTC teeth whitening products. It is a powerful oxidizing agent and depending on conditions hydrogen peroxide can be converted to more potent free radical species. However, development of teeth bleaching products that maintain efficacy over the product shelf-life can be quite challenging. H₂O₂ is a strong oxidizing agent and tends to react with ingredients that are in the formulation that can adversely affect both the efficacy as well as the aesthetic profile of the product. Therefore, product developers and formulators prefer to use anhydrous forms of hydrogen peroxide.

Urea peroxide is a crystalline solid that is commonly used in many bleaching products. Urea peroxide has been shown to provide improved product stability and maintain whitening efficacy compared to hydrogen peroxide solution when used at the equivalent peroxide levels. Nevertheless, studies have also shown that bleaching products containing urea peroxide are not immune from stability issues. Products containing urea peroxide suffer from a low shelf life and often require a cold temperature storage condition and must be stored in a package with excellent moisture barrier property. More stable anhydrous peroxides bleaching materials such as metal salts of percarbonates, perphosphates and persulphates have been used as bleaching agents, however their use in oral care products have been limited by safety concerns and their undesirable organoleptic properties.

Further, amino acid hydrogen peroxide adducts are also known to be used as teeth whitening agent in the oral care products.

Japanese Patent Publication No. 2021138691A describes a peptide-hydrogen peroxide addition product capable of stably holding hydrogen peroxide. The peptide is a dipeptide or a peptide having 6 or more peptide bonds.

U.S. Pat. No. 3,140,149 teaches an improved method for the preparation of perhydrate compounds containing water of crystallization. The perhydrate compound are prepared by reacting an inorganic salt capable of binding water of crystallization such as borates, phosphates, silicates, carbonates and salts of metals such as sodium, potassium, magnesium with hydrogen peroxide and water. The perhydrate compound is useful as bleaching, washing and penetration agents.

U.S. Pat. No. 3,650,705 teaches a potassium pyrophosphate peroxyhydrates containing at least 30 weight percent bound hydrogen peroxide. The peroxyhydrates are prepared by forming a saturated solution of potassium pyrophosphate in concentrated hydrogen peroxide and crystallizing out the product from the saturated solution. The peroxyhydrates are useful as bleaching agents, detergent additives, disinfection agents, and the like.

U.S. Pat. No. 3,860,694 teaches a process for producing perhydrates of sodium carbonate and phosphate. The process includes contacting the precursor of the perhydrate with concentrated hydrogen peroxide solution in such quantity as to develop the desired active oxygen content in the particles. The perhydrates are recovered by drying.

GB Patent No. 2,281,070 teaches a method for preparing peroxyhydrate of condensed phosphates, such as tetrasodium pyrophosphate tris peroxyhydrates. The method involves adding tetra alkali metal pyrophosphate to an aqueous hydrogen peroxide followed by evaporating water from the resulting paste sufficiently to form a dry product. The peroxyhydrates are effective oxidizing bleaches finding applications in detergent formulations such as laundry detergents and dishwashing powders.

Similarly, U.S. Pat. No. 5,739,095 describes a bleaching composition which is a reaction product of hydrogen peroxide and an alkali metal phosphate salt capable of forming a peroxyhydrate with the hydrogen peroxide. The solid bleaching composition is used for ware-washing, laundry, or general hard surface cleaning. The bleaching composition as described in the aforesaid US patent readily dissolves in hot water and is resistant to loss of valuable bleaching oxygen.

U.S. Pat. No. 5,820,841 describes a method of making inorganic salt-hydrogen peroxide complexes, such as, the complexes of phosphates or condensed phosphates or silicate salts of sodium, potassium, calcium or magnesium with hydrogen peroxide. The hydrogen peroxide complex is used for sterilizing medical instruments.

U.S. Pat. No. 6,815,408 teaches a method of cleaning articles such carpets using a composition of hydrogen peroxide and tri-potassium phosphate.

U.S. Pat. No. 4,119,660 teaches a method for making aliphatic diperoxyacids. The method mainly involves adding a dibasic acid having from 10 to 20 carbon atoms to a solution of hydrogen peroxide, sulfuric acid and water.

To promote oral health, a variety of substances have been utilized individually and in combination. It would be highly desirable to have an adduct for optimal improvement of oral health. Therefore, there is a continuous need in the art for hydrogen peroxide containing anhydrous oral care composition which is affordable, sustainable, effective and shelf stable for everyday consumer usage.

### SUMMARY OF THE INVENTION

In one aspect, the present disclosure provides an adduct of (i) peroxide; (ii) alkali metal pyrophosphate, a stabilizing agent; and (iii) at least one compound selected from the group consisting of polyamino acids, proteins, and any combinations thereof. In one embodiments of the present disclosure, the peroxide is hydrogen peroxide or urea peroxide. In another embodiment of the present disclosure, the polyamino acid is polyaspartic acid or polyhistidine, or salts thereof.

In another aspect, the present disclosure provides a composition comprising the adduct of the present disclosure. In one embodiment of the present disclosure, the composition is a nutrition composition, a food composition, a beverage composition, a pharmaceutical composition, a laundry or cleaning composition, a coating composition, a biocide composition, a construction composition, an energy composition, an industrial composition, an oilfield composition, an oral care composition, a skin care composition, an acne treatment composition, a hair care composition, a scalp care composition, a household composition, a performance composition, an agricultural composition, a cosmetic composition, a medical device, a pesticide composition, a veterinary composition, a fuel composition, a lubricant composition, an adhesive composition, a textile composition, an ink composition, a membrane composition, or a disinfectant composition. In one non-limiting embodiment of the present disclosure, the composition is an oral care composition.

In yet another aspect, the present disclosure provides an oral care composition comprising (i) the adduct of present disclosure; (ii) one or more orally acceptable excipients; and (iii) one or more oral care active ingredients. In one non-limiting embodiment of the present disclosure, the oral care composition is a non-aqueous composition comprising water content in the range of from about 0.0% w/w to about 5.0% w/w.

In a still another aspect, the present disclosure provides an oral care composition comprising: (i) from about 0.5 wt. % to about 95.0 wt. % adduct of the present disclosure comprising (a) hydrogen peroxide; (b) sodium pyrophosphate; and (iii) polyaspartic acid or polyhistidine, or salts thereof; and (ii) from about 10.0 wt. % to about 80.0 wt. % one or more orally acceptable excipients; and (iii) from about 0.1 wt. % to about 20.0 wt. % one or more oral care active ingredients.

Another aspect of the present disclosure provides a process for preparing an adduct comprising the steps of: (a) dispersing or dissolving about 0.1 wt. % to 99.0 wt. % alkali metal pyrophosphate in an aqueous peroxide solution at a temperature in the range of from about 4° C. to about 90° C. to obtain a solution or a dispersion; (b) dispersing or dissolving from about 0.1 wt. % to about 99.9 wt. % at least one compound selected from the group consisting of polyamino acids, proteins, and any combinations thereof in the solution or dispersion of process step (a) at a temperature in the range of from about 4° C. to 90° C. under continuous stirring to obtain a solution or dispersion; (c) stirring the resultant solution or dispersion of step (b) from 1.0 rpm to 10,000 rpm and at a temperature of from 0° C. to 90° C.; (d) filtering the resulting slurry or solution of step (c); and/or (e) drying the resultant slurry or solution of step (d) at cold, room or higher temperature to form a desired adduct.

Still another aspect of the present disclosure provides a method to improve oral health wherein the method comprising applying an effective amount of the oral composition of the present disclosure to an oral cavity surface, wherein the method is effective (i) to reduce or inhibit formation of dental caries, (ii) for preventing the demineralization of the teeth, (iii) to promote remineralization of the teeth, (iv) to reduce or inhibit hypersensitivity of the teeth, (v) to treat gingivitis, erosion, cavities, calculus, inflammation, staining and plaque accumulation, and (vi) to reduce biofilm formation, (vii) enhance antibacterial activity, (viii) tooth whitening, and (xi) improve flavor retention.

Yet another aspect of the present disclosure a method for whitening teeth wherein the method comprising administering an oral care composition of the present disclosure to the tooth surface.

### DETAILED DESCRIPTION OF THE INVENTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is/are capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art.

All patents, published patent applications, and non-patent publications mentioned in the specification are indicative of the level of skill of those skilled in the art to which this present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, and/or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combinations of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “amino acid” refers to naturally occurring and synthetic amino acids encompasses the L or D or DL stereoisomers of each amino acid. The non-limiting amino acids includes D-, L- or DL-forms of lysine, arginine, histidine, glycine, serine, threonine, asparagine, glutamine, cysteine, selenocysteine, proline, alanine, valine, isoleucine, leucine, methionine, phenylalanine, tyrosine, tryptophan, aspartic acid, and glutamic acid.

As used herein the term, “peroxides” or “peroxide compounds” refers to a class of compounds that contain a simple oxygen-oxygen bond (—O—O—, peroxy or peroxo moiety group) or a peroxide anion. Suitable and non-limiting examples of the peroxides can include, but are not limited to, hydrogen peroxide, organic peroxides, inorganic peroxides, peroxide complexes, and other compounds containing the peroxy or peroxo group which generate peroxide species in-situ. The inorganic peroxides can include, but are not limited to, alkali metal peroxides, alkaline earth metal peroxides, alkali metal percarbonates, alkaline earth metal percarbonates, ammonium percarbonate, zinc percarbonate, alkali metal persulfates, alkaline earth metal persulfates, ammonium persulfate, zinc persulfate, and the like. Further, suitable and non-limiting examples of the inorganic peroxide can include, but are not limited, sodium peroxide, potassium peroxide, lithium peroxide, calcium peroxide, magnesium peroxide, barium peroxide, strontium peroxide, sodium percarbonate, potassium percarbonate, calcium percarbonate, magnesium percarbonate, barium percarbonate, zinc percarbonate, sodium persulfate, potassium persulfate, calcium persulfate, magnesium persulfate, barium persulfate and zinc persulfate. Similarly, suitable and non-limiting examples of the organic peroxide can include urea peroxides, mono- or poly-peroxides, a combination of a C₁-C₄ alkanol oxidase and a C₁-C₄ alkanol (such as methanol oxidase and ethanol as described in WO 95/07972), alkyl hydroxy peroxides, such as cumene hydroperoxide and t-butyl hydroperoxide.

The term “adduct” refers to a substance wherein peroxide is stably attached to polyamino acids and alkali metal pyrophosphates either covalently or by hydrogen bonding, forming a polyamino acid-alkali metal pyrophosphate-peroxide adduct.

The term “polyamino acid-alkali metal pyrophosphate-peroxide adducts” refers to adducts consisting of polyamino acids or their salts, alkali metal pyrophosphates, and peroxide which are joined together either by covalent bond or a hydrogen bond. Suitable and non-limiting examples of such adducts in the context of the present disclosure include adducts; polylysine-alkali metal pyrophosphate-peroxide adducts, polyarginine-alkali metal pyrophosphate-peroxide adducts-polyhistidine-alkali metal pyrophosphate-peroxide adducts, polyglycine-alkali metal pyrophosphate-peroxide adducts, polyserine-alkali metal pyrophosphate-peroxide adducts, polythreonine-alkali metal pyrophosphate-peroxide adducts, polyasparagine-alkali metal pyrophosphate-peroxide, polyglutamine-alkali metal pyrophosphate-peroxide adducts, polycysteine-alkali metal pyrophosphate-peroxide adducts, polyselen alkali metal pyrophosphate-peroxide adducts, polyproline-alkali metal pyrophosphate-peroxide adducts, polyalanine-alkali metal pyrophosphate-peroxide adducts, polyvaline-alkali metal pyrophosphate-peroxide adducts, polyisoleucine-alkali metal pyrophosphate-peroxide adducts, polyleucine-alkali metal pyrophosphate-peroxide adducts, polymethionine-alkali metal pyrophosphate-peroxide adducts, polyphenylalanine-alkali metal pyrophosphate-peroxide adducts, polytyrosine-alkali metal pyrophosphate-peroxide adducts, polytryptophan-alkali metal pyrophosphate-peroxide adducts, polyaspartic acid-alkali metal pyrophosphate-peroxide adducts, or polyglutamic acid-alkali metal pyrophosphate-peroxide adducts. Further, the peroxide can include any peroxides as herein above described, for example, hydrogen peroxide or urea peroxide, or combinations thereof.

In one aspect, the present disclosure provides adducts or complexes of peroxides, alkali metal pyrophosphates, and at least one compound selected from the group consisting of polyamino acids including their salts, proteins, and any combinations thereof.

The peroxide according to the present disclosure can be any peroxide known to those skilled in the relevant art. The peroxides suitable for the purpose of the present disclosure can be organic or inorganic peroxide(s). Suitable examples of such inorganic peroxides can include, but are not limited to, alkali metal peroxides such as lithium peroxide, sodium peroxide, potassium peroxide; alkaline earth metal peroxides such as calcium peroxide, magnesium, strontium peroxide, and barium peroxide; alkali metal percarbonates such as sodium percarbonate, potassium percarbonate; alkaline earth metal percarbonates such as calcium percarbonate, magnesium percarbonate, barium percarbonate; ammonium percarbonate, zinc percarbonate, alkali metal persulfates such as sodium persulfate, potassium persulfate; alkaline earth metal persulfates such as calcium persulfate, magnesium persulfate, and barium persulfate; ammonium persulfate, zinc persulfate, and the like. Similarly, suitable and non-limiting examples of the organic peroxide can include urea peroxides, mono- or poly-peroxides, a combination of a C₁-C₄ alkanol oxidase and a C₁-C₄ alkanol (such as methanol oxidase and ethanol as described in WO 95/07972), alkyl hydroxy peroxides, such as cumene hydroperoxide and t-butyl hydroperoxide. In one non-limiting embodiment of the present disclosure, the peroxide can be a hydrogen peroxide or urea peroxide, or any combinations thereof.

The alkali metal pyrophosphate used in the adducts of the present disclosure can include, but are not limited to, one-, two- or trisubstituted potassium or sodium pyrophosphate, disubstituted alkali metal pyrophosphates, 4-substituted alkali metal pyrophosphates, and mixtures thereof. In one non-limiting embodiment of the present disclosure, the alkali metal pyrophosphate can be selected from the group consisting of sodium orthophosphate, sodium dihydrogen diphosphate (Na₂H₂P₂O₇), dipotassium pyrophosphate, sodium pyrophosphate (Na₄P₂O₇), potassium pyrophosphate (K₄P₂O₇), and mixtures thereof. Further, the alkali metal salts are useful in both their hydrated and unhydrate forms. In one non-limiting embodiment of the present disclosure, the alkali metal pyrophosphate is sodium pyrophosphate.

The adduct according to the present disclosure comprises at least one compound selected from the group consisting of polyamino acids including their salts, proteins, and any combinations thereof. In one non-limiting embodiment of the present disclosure, the compound can be a polyamino acid. The polyamino acids according to the present disclosure refers to compounds having at least one peptide bond. The peptide bond, also referred to as an amide bond, is formed by the reaction between the alpha-carboxyl group of one amino acid and the alpha-amino group of a second amino acid. The polyamine acids suitable for use in the present disclosure can comprise two or more amino acids joined by peptide bonds. In one non-limiting embodiment of the present disclosure, the number of amino acids joined by peptide bond in the polyamino acid can vary in the range of from about 2 to 10,000. In one non-limiting embodiment of the present disclosure, the number of amino acids joined by peptide bonds can vary in the range of from about 2 to 100, or from about 100 to 1000, or from about 1000 to 10,000. The polyamino acids according to the present disclosure may thus be classified as peptides or oligopeptides, and polypeptides depending on the number of amino acids linked by peptide bonds or amide bond in the polyamino acids. The polyamino acids including the peptides, oligopeptides, and polypeptides are long, unbranched chains of peptides up to approximately 10,000 amino acids Further, the terms “polypeptides” and “polyamino acids” can be used interchangeably in the context of the present disclosure.

Accordingly, the polyamino acids according to the present disclosure can include, but are not limited to, polylysine, polyarginine, polyhistidine, polyglycine, polyserine, polythreonine, polyasparagine, polyglutamine, polycysteine, polyselenocysteine, polyproline, polyalanine, polyvaline, polyisoleucine, polyleucine, polymethionine, polyphenylalanine, polytyrosine, polytryptophan, polyaspartic, polyglutamic acids or salts thereof, and any combinations thereof.

In another non-limiting embodiment of the present disclosure, the compound can be protein. Proteins are well known in the art and refers to large macromolecular peptide compounds formed from one or more polypeptide chains joined together. Proteins are thus large peptides containing 50 or more amino acids. The terms “peptide” and “protein” are commonly confused. Not all peptides form proteins, but all proteins consist of peptides. Proteins are large peptides (polypeptides) containing 50 or more amino acids or molecules that consist of multiple peptide subunits. Also, proteins typically display more complex structure than simpler peptides.

The adduct according to present disclosure can comprises (i) from about 1.0 wt. % to about 50.0 wt. % of peroxide; (ii) from about 0.1 wt. % to about 99.0 wt. % of alkali metal pyrophosphate; and (iii) from about 0.1 wt. % to about 99.0 wt. % of at least one compound selected from the group consisting of polyamino acids, proteins, and any combinations thereof. In one non-limiting embodiment of the present disclosure, the peroxide can be present in an amount of from about 1.0 wt. % to about 10.0 wt. %, or from about 10.0 wt. % to about 20.0 wt. %, or from about 20.0 wt. % to about 30.0 wt. %, or from about 30.0 wt. % to about 40.0 wt. %, or from about 40.0 wt. % to about 50.0 wt. %, based on the total adduct weight.

In another non-limiting embodiment of the present disclosure, the amount of alkali metal pyrophosphate can vary in the range of from about 0.1 wt. % to about 10.0 wt. %, or from about 10.0 wt. % to about 20.0 wt. %, or from about 20.0 wt. % to about 30.0 wt. %, or from about 30.0 wt. % to about 40.0 wt. %, or from about 40.0 wt. % to about 50.0 wt. %, or from about 50.0 wt. % to about 60.0 wt. %, or from about 60.0 wt. % to about 70.0 wt. %, or from about 70.0 wt. % to about 80.0 wt. %, or from about 80.0 wt. % to about 90.0 wt. %, or from about 90.0 wt. % to about 99.0 wt. %, based on the total adduct weight.

In yet another non-limiting embodiment of the present disclosure, the polyamino acids or proteins can be present in an amount of from about 0.1 wt. % to about 10.0 wt. %, or from about 10.0 wt. % to about 20.0 wt. %, or from about 20.0 wt. % to about 30.0 wt. %, or from about 30.0 wt. % to about 40.0 wt. %, or from about 40.0 wt. % to about 50.0 wt. %, or from about 50.0 wt. % to about 60.0 wt. %, or from about 60.0 wt. % to about 70.0 wt. %, or from about 70.0 wt. % to about 80.0 wt. %, or from about 80.0 wt. % to about 90.0 wt. %, or from about 90.0 wt. % to about 99.0 wt. %, based on the total adduct weight.

Further, the adduct according to the present disclosure can have a pH of about 4 to about 10, or from about 4 to 8, or from about 5 to 8.

The adduct according to the present disclosure can further be used in different compositions depending on end use applications. In a still another aspect, the present disclosure provides a composition comprising the present adduct. The compositions can include, but are not limited to, a nutrition composition, a food composition, a beverage composition, a pharmaceutical composition, a laundry or cleaning composition, a coating composition, a bio-cide composition, a construction composition, an energy composition, an industrial composition, an oilfield composition, an oral care composition, a skin care composition, a hair care composition, an acne treatment composition, a scalp care composition, a household composition, a performance composition, an agricultural composition, a cosmetic composition, a medical device, a pesticide composition, a veterinary composition, a fuel composition, a lubricant composition, an adhesive composition, a textile composition, an ink composition, a membrane composition, or a disinfectant composition.

In another non-limiting embodiment of the present disclosure, the composition can be an oral care composition. The oral care composition according to the present disclosure can comprise (i) the adduct of present disclosure; (ii) one or more orally acceptable excipients; and (iii) one or more oral care active ingredients.

The orally acceptable excipients used in the present oral care composition can be selected from the group consisting of thickening agents, desensitizing agents, whitening agents, tartar control agents, abrasives, binders, detergents, adhesion agents, foam modulators, pH modifying agents, mouth feel agents, sweeteners, flavorants, colorants, preservatives, humectants, fluoride containing salts, encapsulants and compounds, water, polymers, orally acceptable surfactants, stain preventors and mixtures thereof.

Similarly, the one or more oral care active ingredients in the oral care composition of the present disclosure can be selected from the group consisting of an analgesic, an antibacterial, an anticalculus agents, an antibiotic, a probiotic, an antioxidant, a peptide, an enzyme, a cooling agent, a preservative, a desensitizing agent, a dental remineralization agent, odor or breath freshening agents, warming agents, herbal agents, medicaments, vitamins, taste masking agents, pharmaceutical agent, a therapeutic agent, vitamin, a mineral, warming agent, a sensate, throat-soothing agent, spices, caffeine, drug, and mixtures thereof.

Suitable and non-limiting examples of antibacterial agents can include zinc ion sources such as zinc acetate, zinc citrate, zinc gluconate, zinc glycinate, zinc oxide, zinc sulfate, and sodium zinc citrate; phthalic acid and salts thereof such as magnesium monopotassium phthalate; hexetidine; octenidine; sanguinarine; benzalkonium chloride; domiphen bromide; alkylpyridinium chlorides such as cetylpyridinium chloride (CPC) (including combinations of CPC with zinc and/or enzymes), tetradecylpyridinium chloride and N-tetradecyl-4-ethylpyridinium chloride; iodine; sulfonamides, bisbiguanides such as alexidine, chlorhexidine and chlorhexidine; digluconate; piperidino derivatives such as delmopinol and octapinol, magnolia extract, grapeseed extract, raspberry ketone, menthol, geraniol, citral, eucalyptol, eugenol, stannous fluoride, sodiummonomagnolia bark extracts, Chinese traditional medicines, thymol, 4-isopropyl m-cresol (IPMP, o-cymen 5-ol) antibiotics such as amoxicillin, tetracycline, doxycycline, minocycline, metronidazole, neomycin, kanamycin and clindamycin, and the like. A further illustrative list of useful antibacterial agents is provided in U.S. Pat. No. 5,776,435 to Gaffar et al.

Suitable flavorants include those flavors known to the skilled artisan, such as natural and artificial flavors. These flavorings chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also, useful flavorings are artificial, natural and synthetic fruit flavors such as vanilla, and citrus oils including lemon, orange, lime, grapefruit, and fruit essences including apple, pear, peach, grape, blueberry, strawberry, raspberry, cherry, plum, pineapple, apricot and so forth. These flavoring agents can be used in liquid or solid form and can be used individually or in admixture. Commonly used flavors include mints such as peppermint, menthol, spearmint, artificial vanilla, cinnamon derivatives, and various fruit flavors, whether employed individually or in admixture.

Flavors can also provide breath freshening properties, particularly the mint flavors when used in combination with the cooling agents, described herein below. Other useful flavorings include aldehydes, esters and ketones such as cinnamyl acetate, cinnamaldehyde, citral diethylacetal, dihydrocarvyl acetate, eugenyl formate, raspberry ketone p-methylamisol, and so forth may be used. Generally, any flavoring or food additive such as those described in Chemicals Used in Food Processing, publication 1274, pages 63-258, by the National Academy of Sciences, may be used. This publication is incorporated herein by reference. This can include natural as well as synthetic flavors.

Non-limiting examples of suitable animal and vegetable oils can include, but are not limited, sunflower oil, corn oil, soy oil, avocado oil, jojoba oil, squash oil, raisin seed oil, sesame seed oil, walnut oil, fish oil, glycerol tricaprocaprylate, purcellin oil, liquid jojoba, and blends thereof. Also suitable are natural oils such as oils of eucalyptus, lavender, vetiver, Litsea cubeba, lemon, sandalwood, rosemary, chamomile, savory, nutmeg, cinnamon, hyssop, caraway, orange, geranium, cade, bergamot, and blends thereof. There are five commonly available vegetable oils which contain greater than 50% linoleic acid groups (as their glycerol esters). These are, in decreasing order of linoleic content: Safflower Oil (75%); Sunflower Oil (67.8%); Corn Oil (56%); Cottonseed Oil (55%) and Soybean Oil (52%).

Non-limiting examples of suitable vitamins or minerals can include vitamin A, vitamin C, vitamin D, vitamin E, vitamin K, vitamin B6, vitamin B12, thiamine, riboflavin, biotin, folic acid, niacin, pantothenic acid, sodium, potassium, calcium, magnesium, iron, copper, zinc, selenium, manganese, choline, chromium, molybdenum, cobalt and combinations thereof, can be used.

Similarly, suitable examples of whitening agents that can be used in the present oral care composition include, but are not limited to, dyes, polyphosphates, phytic acid and its' salts, complexed bleaching or bleaching agents. Suitable bleaching or whitening agents include peracids, peroxide compounds. Peroxides are believed to whiten the teeth by releasing hydroxyl radicals capable of breaking down the plaque-stain complex into a form that can be flushed away or removed by abrasives.

Further, a film-forming polymer can be included in the present oral care composition. Suitable examples of such film forming polymers can include, but are not limited, to synthetic anionic polymeric polycarboxylate (SAPP), such a PVM/MA copolymer (Gantrez S-97, Ashland Inc.). Such polymers are also described in the U.S. Pat. Nos. 5,334,375 and 5,505,933. SAPP's have previously been described as useful for dentin sensitivity reduction. Moreover, SAPP's have previously been described as antibacterial-enhancing agents, which enhance delivery of an antibacterial agent to oral surfaces, and which enhance the retention of the antibacterial agent on oral surfaces. It is well within the contemplation of the present invention that film-forming polymers, such as PVM/MA copolymer, can be employed in the compositions of the present invention as a means of reducing stain formation.

Suitable examples of the sweetening agents can include, but are not limited to, sucrose, glucose, saccharin, dextrose, levulose, lactose, mannitol, sorbitol, fructose, maltose, xylitol, erythritol, saccharin salts, thaumatin, aspartame, D-tryptophan, dihydrochalcones, acesulfame, sucralose and cyclamate salts, especially sodium cyclamate and sodium saccharin, and mixtures thereof.

The oral care composition according to the present disclosure can further comprise abrasives. Suitable and non-limiting examples of such abrasives can include, but are not limited to, silica abrasives, such as standard cleaning silicas, high cleaning silicas or any other suitable abrasive silicas. Additional examples of abrasives that can be used in addition to or in place of the silica abrasives include, for example, a calcium phosphate abrasive, e.g., tricalcium phosphate (Ca₃(PO₄)₂), hydroxyapatite (Ca₁₀(PO₄)₆(OH)₂), or dicalcium phosphate dihydrate CaHPO₄·2H₂O or calcium pyrophosphate; calcium carbonate abrasive; or abrasives such as sodium metaphosphate, potassium metaphosphate, aluminum silicate, calcined alumina, bentonite or other siliceous materials, or combinations thereof. The silica component of the present silica substrate is an amorphous precipitated silica. Precipitated silicas include the following products available from the J. M. Huber Corporation, Edison, N.J.: Zeodent® 103, Zeodent® 113, Zeodent® 114, Zeodent® 115, Zeodent® 118, Zeodent® 119, Zeodent® 165, and Zeodent® 9175.

The oral care composition according to the present disclosure can further comprises suitable anionic surfactants. Suitable examples of such surfactants can include, but are not limited to, water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms, such as sodium lauryl sulfate and sodium coconut monoglyceride sulfonate. Other suitable anionic surfactants are sarcosinates, such as sodium lauroyl sarcosinate, taurates, sodium lauryl sulfoacetate, sodium lauroyl isethionate, sodium lauryl carboxylate, and sodium dodecyl benzenesulfonate, and mixtures thereof. Other useful anionic surfactant includes sarcosinate surfactants, isethionate surfactants and taurate surfactants. Surfactants are alkali metal or ammonium salts of these surfactants, sodium and potassium salts of lauroyl sarcosinate, myristoyl sarcosinate, palmitoyl sarcosinate, stearyl sarcosinate, Amisoft CS-11 (amino acid and L-Glutamic Acid) and oleoyl sarcosinate.

Examples of suitable cationic surfactants are derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride etc.

Examples of suitable nonionic surfactants can include, but are not limited to, Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Examples of suitable zwitterionic surfactants can include, but not limited to derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

In another embodiment of the present disclosure, the oral care composition can further comprise thickening agents. Suitable examples of such thickening agents include, but not limiting to, water-soluble cellulose ethers such as sodium carboxymethylcellulose, sodium carboxymethyl hydroxyethyl cellulose and, hydroxyethyl cellulose, vinylpyrrolidone polymers (also referred to as N-vinylpyrrolidone, N-vinyl-2-pyrrolidone and N-vinyl-2-pyrrolidinone, polyvinyl pyrrolidone) as a monomeric unit. Other suitable thickening agents include carboxyvinyl polymers (carbomers), carrageenan, laponite and other natural gums such as gum karaya, xanthan gum, guar gum, gum arabic, and gum tragacanth. Colloidal magnesium aluminum silicate or finely divided silica can be used as part of the thickening agent to further improve texture. A class of other thickening or gelling agents includes a class of homopolymers of acrylic acid cross-linked with an alkyl ether of pentaerythritol or an alkyl ether of sucrose. In various preferred embodiments, the carrier may comprise polymers and/or copolymers of polyethylene glycol, of ethylene oxide/propylene oxide, and of silicone. If such copolymers/polymers are used, they may be selected from commercially available materials. Block copolymers of ethylene oxide propylene oxide are useful, but higher molecular weight, e.g., >5000 Da are preferred, e.g. including PLURA CARE® L1220 (available from BASF, Wyandotte, Mich., United States of America). Low or medium molecular weight polyethylene glycol, e.g., PEG 400, PEG 600, PEG 800, PEG 1000 and mixtures thereof are also useful. It is preferred that the carrier(s) provide a dentifrice with a viscosity of about 10,000 CPS to about 700,000 CPS, preferably about 30,000 CPS to about 300,000 CPS.

In another embodiment, the present application, anti-microbial agents can include, but not limited to halogenated diphenyl ether, 2,4,4′-trichloro-2′-hydroxy-diphenyl ether, 2,2′-dihydroxy-5,5′-dibromo-diphenyl ether, 2,2′-methylenebis-4(4-chloro-6-bromo-phenol), halogenated salicylanilides and halogenated cabanilides, stannous chloride, zinc lactate, zinc citrate, zinc oxide.

As used herein “fluoride-providing compound” can include, but are not limited to, sodium fluoride, potassium fluoride, amine fluoride, ammonium fluoride, lead fluoride, manganese fluoride, a copper fluoride such as cuprous fluoride, zinc fluoride, barium fluoride, sodium fluorosilicate, ammonium fluorosilicate, sodium fluorozirconate, sodium monofluorophosphate, potassium fluorozirconates, sodium monofluorophosphate, aluminum mono- and di-fluorophosphate, and fluorinated sodium calcium pyrophosphate, a tin fluoride such as stannous fluoride, stannic fluoride or stannous chlorofluoride, and sodium hexafluorostannate.

In another non-limiting embodiment of the present disclosure, the composition can further comprises at least one stabilizing agent for the stabilization of hydrogen peroxide, such as, alkali metal stannates, phosphoric acid, sodium citrate, colloidal silicates, organophosphonates, sodium phenolsulfate; sodium stannate; N,N-lower alkyl aniline, sulfamic acid, sulfolane, and dinormal lower alkyl sulfones and sulfoxides; phosphonic acids and their salts; acrylic acid polymers; pyrophosphates, polyphosphates; 2-Anilinoethanol, antioxidant and free radical scavenger; BHT (butylated hydroxytoluene), polyphenols, peptides or polymers, polyamino polyphosphonic acids and/or their salts.

Further, the oral care composition according to the present disclosure can be formulated as a toothpaste, tooth gel, subgingival gel, tooth powder, mouth-rinse, mouth wash, denture cream, denture powder, mouth-spray, chewable tablet, dissolvable film, strip or chewing gum. In one non-limiting embodiment of the present disclosure, the oral care composition can be a toothpaste. In another non-limiting embodiment of the present disclosure, the oral care composition can be a tooth gel. The oral care composition according to the present disclosure is a non-aqueous composition, mean the total amount of water that can be present in the oral care composition of the present disclosure does not exceed 5.0% w/w. In one non-limiting embodiment of the present disclosure, the oral care composition comprises water content in the range of from about 0.0% to about 5.0% w/w. Further, pH of the present oral care composition can vary in the range of from about 4 to about 10, or from about 4 to 8, or from about 5 to 8.

The oral care composition according to present disclosure can comprise (i) from about 0.5 wt. % to about 95.0 wt. % adduct of the present disclosure; (ii) from about 10.0 wt. % to about 80.0 wt. % one or more orally acceptable excipients; and (iii) from about 0.1 wt. % to about 20.0 wt. % one or more oral care active ingredients. In one non-limiting embodiment of the present disclosure, the adduct can be present in an amount of from about 0.5 wt. % to about 25.0 wt. %, or from about 25.0 wt. % to about 50.0 wt. %, or from about 50.0 wt. % to about 75.0 wt. %, or from about 75.0 wt. % to about 95.0 wt. %, based on the total weight of the oral care composition. In some embodiments, the suitable range of adduct employed in the oral care composition of the present disclosure can be varied from about 0.5 wt. % to about 0.1 wt. %; or from about 0.1 wt. % to about 1 wt. %; or from about 1 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. % or from about 90 wt. % to about 95 wt. % based on the total weight of the oral care composition.

Similarly, the suitable range of orally acceptable excipients in the present oral care composition can vary from about 10.0 wt. % to about 15.0 wt. %; or from about 15.0 wt. % to about 20.0 wt. %; or from about 20.0 wt. % to about 25.0 wt. %; or from about 25.0 wt. % to about 30.0 wt. %; or from about 30.0 wt. % to about 35.0 wt. %; or from about 35.0 wt. % to about 40.0 wt. %; or from about 40.0 wt. % to about 45.0 wt. %; or from about 45.0 wt. % to about 50.0 wt. %; or from about 50.0 wt. % to about 55.0 wt. %; or from about 55.0 wt. % to about 60.0 wt. %; or from about 60.0 wt. % to about 65.0 wt. %; or from about 65.0 wt. % to about 70.0 wt. %; or from about 70.0 wt. % to about 75.0 wt. %; or from about 75.0 wt. % to about 80.0 wt. % based on the total weight of the oral care composition.

In another non-limiting embodiment of the present disclosure, the suitable range of oral care active ingredient can vary from about 0.1 wt. % to about 1.0 wt. %; or from about 1.0 wt. % to about 2.5 wt. %; or from about 2.5 wt. % to about 5.0 wt. %; or from about 5.0 wt. % to about 10.0 wt. %, or 10.0 wt. % to about 15.0 wt. %; or from about 15.0 wt. % to about 20.0 wt. %, based on the total weight of the oral care composition.

Another aspect of the present disclosure provides a process for preparing the adduct of the present disclosure. In one non-limiting embodiment of the present disclosure, the process comprising: (a) dispersing or dissolving about 0.1 wt. % to about 99.0 wt. % of at least one alkali metal pyrophosphate in a hydrogen peroxide solution at a temperature in the range of from about 4° C. to about 90° C. to obtain a solution or a slurry; (b) dispersing or dissolving from about 0.1 wt. % to 99.0 wt. % at least one compound selected from the group consisting of polyamino acids including their salts, proteins, and any combinations thereof in the solution of process step (a) at a temperature in the range of from about 30° C. to 50° C. under continuous stirring to obtain a solution or slurry; (c) stirring the resultant solution or slurry of step (b) from 1 rpm to 10,000 rpm and at a temperature of about 0° C. to 90° C.; (d) drying the resultant slurry or solution of step (c) at cold, room or higher temperature to form a desired adduct in solid form.

The hydrogen peroxide solution used in the process of the present disclosure is an aqueous solution of hydrogen peroxide with an active peroxide concentration in the range of from about 20.0 wt. % to about 80.0 wt. %. In one non-limiting embodiment of the present disclosure, the active peroxide content can vary in the range of from about 20.0 wt. % to about 30.0 wt. %; or from about 30.0 wt. % to about 40.0 wt. %, or from about 40.0 wt. % to about 50.0 wt. %; or from about 50.0 wt. % to about 60.0 wt. %; or from about 60.0 wt. % to about 70.0 wt. %; or from about 70.0 wt. % to about 80.0 wt. %.

Any suitable drying methods known in the related art can be used for the purpose of drying the resultant solution or slurry in the process step (d) of the present process. Suitable examples of such drying methods can include, but are not limited to, fluidized bed drying, spray drying, freeze drying, wet and dry granulation, dry and wet compacting methods, belt drying, drum drying, granulation, flash mill drying, flash jet drying, agglomeration, and the like.

The adduct obtained in accordance with the present process can be milled further to obtain adduct in fine powdery form. The milling can be carried out to obtained fine powder adducts with desired particle size distribution.

In another embodiment of the present disclosure, the present disclosure provides a method to improve oral health. The method comprises applying an effective amount of the oral care composition of the present disclosure to an oral cavity surface wherein the method is effective (i) to reduce or inhibit formation of dental caries, (ii) for preventing the demineralization of the teeth, (iii) to promote remineralization of the teeth, (iv) to reduce or inhibit hypersensitivity of the teeth, (v) to treat gingivitis, erosion, cavities, calculus, inflammation, staining and plaque accumulation, and (vi) to reduce biofilm formation, (vii) enhance antibacterial activity, (viii) tooth whitening, and (xi) improve flavor retention.

In another non-limiting embodiment of the present disclosure, the method to improve oral health is effective at delivering and retaining therapeutic agents such as (i) antibacterial, (ii) anti-caries, (iii) anti-gingivitis, (iv) anti-inflammation, (v) anti-biofilm, (vi) anti-staining, (vii) anti-erosion, and the like.

Another aspect of the present disclosure also provides a method for whitening teeth, wherein the method comprises administering the oral care composition of the present disclosure to the teeth surface.

The oral care composition comprising the polyamino acid-alkali metal pyrophosphate-peroxide adduct according to the present disclosure is stable for a long period of time and has high efficiency in bleaching of tooth and improves oral health.

Further, certain aspects of the present application are illustrated in detail by way of the following examples. The examples are given herein for illustration of the application and are not intended to be limiting thereof.

### EXAMPLES

### Test Methods:

Unless indicated otherwise, the following test methods were utilized in the Examples that follows:

### Example 1: Preparation of Adducts of Polyamino Acids-Alkali Metal Pyrophosphate and Peroxides

25.0 g tetra sodium pyrophosphate and 25.0 g polyaspartic acid sodium salt were dissolved in 100 ml. 35% hydrogen peroxide solution under continuous stirring at room temperature. The obtained solution was then dried in an oven at 60° C. for 18 hrs. and a solid material was obtained. The solid material was then milled to fine powder. Calcium pyrophosphate was added to the powder as an anti-caking agent. The obtained fine powder has 24 wt. % hydrogen peroxide.

TABLE 1
Polyaspartic acid sodium salt -tetrasodium pyrophosphate-
H₂O₂ adducts and H₂O₂ payloads.

| | H₂O₂ payload |
|---|---|
| Adduct composition | (w/w %) |
| Polyaspartic acid sodium salt -tetrasodium | 23 |
| pyrophosphate (70:30)-H₂O₂ adduct | |
| Polyaspartic acid sodium salt -tetrasodium | 24 |
| pyrophosphate (50:50)-H₂O₂ adduct | |
| Polyaspartic acid sodium salt -tetrasodium | 24 |
| pyrophosphate (40:60)-H₂O₂ adduct | |
| Polyaspartic acid sodium salt -tetrasodium | 25 |
| pyrophosphate (30:70) - H₂O₂ adduct | |
| Polyaspartic acid sodium salt -tetrasodium | 26 |
| pyrophosphate (20:80) - H₂O₂ adduct | |
| Polyaspartic acid sodium salt -Histidine (80:20) - | 18 |
| H₂O₂ adduct | |
| Polyaspartic acid sodium salt -Na Carbonate | 25 |
| (60:40)-H₂O₂adduct | |
| 1-Histidine-H₂O₂ adduct | 18 |
| Polyaspartic acid sodium salt-H₂O₂ adduct | 19 |

### Example 2: Adducts Shelf-Life Stability

The adducts of Example 1 were stored in jars for 12 weeks at 40° C. Hydrogen peroxide contents were determined using permanganate titration.

TABLE 2
Stability of Adducts of Example 1 Aged for 12 weeks at 40° C.

| | | H₂O₂ payload |
|---|---|---|
| | Initial H₂O₂ | after 12 weeks |
| | payload | aging at 40° C. |
| Adduct composition | (% w/w) | (% w/w) |
| PolyAspartic acid sodium salt -tetra Na | 19 | 14 |
| pyrophosphate (70:30)- H₂O₂ adduct | | |
| PolyAspartic acid sodium salt -tetra Na | 24 | 21 |
| pyrophosphate (50:50) - H₂O₂ adduct | | |
| PolyAspartic acid sodium salt -tetra Na | 24 | 21 |
| pyrophosphate (40:60) - H₂O₂ adduct | | |
| PolyAspartic acid sodium salt -tetra Na | 25 | 21 |
| pyrophosphate (30:70) - H₂O₂ adduct | | |
| PolyAspartic acid sodium salt -tetra Na | 26 | 24 |
| pyrophosphate (20:80) - H₂O₂ adduct | | |
| Polyaspartic acid sodium salt -Histidine | 18 | 15 |
| (80:20) - H₂O₂ adduct | | |
| 1-Histidine- H₂O₂ adduct | 18 | 17 |
| Polyaspartic acid sodium salt-- H₂O₂ | 19 | 12 |
| adduct | | |

### Example 3: In Vitro Tooth Whitening Evaluations of Polyaspartic Acid Sodium Salt-Tetra Sodium Pyrophosphate (40:60)-H₂O₂ Adduct and PVP—H₂O₂ Adduct

Polyaspartic acid sodium salt-tetrasodium pyrophosphate (40:60)-H₂O₂ adduct was dissolved in 100 ml deionized water to make a 3% w/w H₂O₂ solution. PVP—H₂O₂ adduct solution was also prepared and adjusted to 3% w/w hydrogen peroxide solution and used as a positive control. Tea and coffee-stained bovine teeth were treated with the polyaspartic acid sodium salt-tetrasodium pyrophosphate (40:60)-H₂O₂ adduct or PVP—H₂O₂ adduct solutions for 2 min, 5 min and 10 min respectively at 37° C. The whitening efficacies of adducts solutions were determined for each treatment time. The color change (ΔE) for each bovine tooth was determined by measuring the color before and after treatment using a handheld CM-2600d spectrophotometer. Changes in color was assessed using ΔE, where

$$Δ E = \sqrt{{( {L}_{2} - {L}_{1} )}^{2} + {( {a}_{2} - {a}_{1} )}^{2} + {( {b}_{2} - {b}_{1} )}^{2}}$$

The values L, a, and b indicate the black/white, green/red, and blue/yellow axes of the color space, respectively. Result showed that the solutions containing polyaspartic acid sodium salt-tetrasodium pyrophosphate (40:60)-H₂O₂ adduct removed significantly more stains from the bovine teeth compared to PVP— H₂O₂ adduct solutions at 3% H₂O₂ concentration as shown in Table 3.

TABLE 3
Tooth Whitening Evaluation Using Stained Bovine Teeth

| | ΔE 2 min. | ΔE 5 min. | ΔE 10 min. |
|---|---|---|---|
| Test solution with 3% H₂O₂ | treatment | treatment | treatment |
| PolyAspartic acid sodium | 6.9 | 14 | 25.4 |
| salt -tetrasodium | | | |
| pyrophosphate (40:60) - | | | |
| H₂O₂ adduct | | | |
| PVP- H₂O₂ adduct | 2.7 | 4.6 | 7.5 |

### Example 4

In this example, stability of polyaspartic acid sodium salt-tetrasodium pyrophosphate (40:60)-H₂O₂ adduct of Example 1 was tested in anhydrous toothpaste formulations. For this, anhydrous toothpaste formulation A was prepared using the ingredients in amounts as listed in Table 4. A. The anhydrous tooth paste formulation A was aged at 40° C. for 2 weeks. Similarly, two different control toothpaste formulations B and C were also prepared using L-Histidine-H₂O₂ adduct and urea peroxide and were also aged at 40° C. for 2 weeks. The stability of the polyaspartic acid sodium salt-tetrasodium pyrophosphate (40:60)-H₂O₂ adduct and urea peroxide were determined using potassium per-manganate titration. Polyaspartic acid sodium salt-tetrasodium pyrophosphate (40:60)-H₂O₂ adduct found to be highly stable in anhydrous toothpaste formulation A compared to urea peroxide.

TABLE 4
Anhydrous Tooth Whitening Toothpaste Formulations

| | Formula- | Formula- | Formula- |
|---|---|---|---|
| Ingredients | tion A | tion B | tion C |
| Calcium pyrophosphate | 15.0 | 15.0 | 15.0 |
| Propylene glycol | QS | QS | QS |
| PEG 400 | 10.0 | 10.0 | 10.0 |
| Flexithix | 0.20 | 0.20 | 0.20 |
| Glycerin | 3.00 | 3.00 | 3.00 |
| Carbopol 974P | 1.00 | 1.00 | 1.00 |
| Polyaspartic acid sodium salt- | 13.0 | 0.0 | 0.0 |
| Tetrasodium Pyrophosphate | | | |
| (40:60)-H₂O₂ adduct | | | |
| L-Histidine-H2O2 adduct | 0.0 | 17.00 | 0 |
| Urea Peroxide | 0.0 | 0.0 | 8.6 |
| Peppermint flavor | 2.25 | 2.25 | 2.25 |
| Sodium lauryl sulfate | 2.00 | 2.00 | 2.00 |
| Silica fumed | 1.50 | 1.50 | 1.50 |
| Mono fluorophosphate (MFP) | 0.76 | 0.76 | 0.76 |
| Sodium Saccharin | 0.60 | 0.60 | 0.60 |
| Phosphoric acid | 0.20 | 0.20 | 0.20 |
| Sucralose | 0.05 | 0.05 | 0.05 |
| Butylated hydroxytoluene | 0.03 | 0.03 | 0.03 |
| Total | 100.0 | 100.0 | 100.0 |

TABLE 5
Hydrogen Peroxide Stability in Anhydrous Toothpastes at 45° C.

| | Initial H₂O₂ | H₂O₂ payload after | Air |
|---|---|---|---|
| | payload | 2 weeks of storage | bubbles |
| Formulations | (% w/w) | at 40° C. (% w/w) | formation |
| Formulation A | 3.1 | 3.0 | minimum |
| Formulation B | 3.0 | 2.9 | minimum |
| Formulation C | 3.2 | 2.2 | significant |

TABLE 6
Low Water Toothpaste Formulation

| Ingredients | % (w/w) |
|---|---|
| Propylene Glycol, USP/NF | QS |
| Water | 5.0 |
| PEG-600 | 7.44 |
| Sodium Mon Fluorophosphate | 0.76 |
| Calcium Pyrophosphate | 30.00 |
| Carbopol 974P | 2.00 |
| Disodium Pyrophosphate | 0.5 |
| Polyaspartic acid sodium salt- | 15 |
| tetrasodium pyrophosphate | |
| (40:60)- H₂O₂ adduct | |
| Fumed Silica | 0.75 |
| SLS, USP/NF | 2.0 |
| Sodium Saccharin | 0.5 |
| BHT | 0.04 |
| Sucralose | 0.05 |
| Menthol | 0.3 |
| Total | 100.0 |

TABLE 7
Teeth Bleaching Gel Formulation

| Ingredients | % (w/w) |
|---|---|
| Glycerin | 2.0 |
| Propylene glycol | QS |
| Carbopol 974P | 0.85 |
| Polyaspartic acid sodium salt- | 30 |
| tetrasodium pyrophosphate | |
| (40:60)- H₂O₂ adduct | |
| Saccharine | 0.5 |
| Menthol | 0.5 |
| Total | 100.0 |

TABLE 8
Teeth Bleaching Strip Formulation

| Ingredients | % (w/w) |
|---|---|
| Glycerin | 2.0 |
| Propylene glycol | QS |
| Carbopol 974P | 0.85 |
| PolyAspartic acid sodium salt- | 30 |
| tetrasodium pyrophosphate | |
| (40:60)- H₂O₂ adduct | |
| Saccharine | 0.5 |
| Backing film | — |
| Total | 100.0 |

### Example 4: Tooth Whitening Tablets

The teeth whitening tablets are produced as follows; the ingredients as shown in Table 8 in powder form are mixed. The ingredients may also be granulated to help them flow through a tablet press. The process involves compacting the ingredients then pulverizing them to a fine consistency. The moisture content should be controlled throughout the process. Lubricants are mixed last, so they coat all the other ingredients well. The prepared mixed powder is then pressed using a rotary tablet press.

TABLE 9
Teeth Whitening Tablet

| Ingredients | % (w/w) |
|---|---|
| Micro crystalline cellulose | 3.00 |
| Sucralose | 0.50 |
| PolyAspartic acid sodium salt- | 20.00 |
| tetrasodium pyrophosphate | |
| (40:60)-H₂O₂ adduct | |
| Mannitol | 35.00 |
| Xylitol | 10.00 |
| Silica | 2.00 |
| Calcium Carbonate | 32.00 |
| Sodium laurate sulphate | 2.30 |
| Magnesium stearate | 0.96 |
| Sodium Fluoride | 0.24 |
| Peppermint micron flavor | 1.00 |
| Total | 100 |

What is claimed is:

1. An adduct of (i) peroxide; (ii) alkali metal pyrophosphates, a stabilizing agent; and (iii) at least one compound selected from the group consisting of polyamino acids, proteins, and any combinations thereof.

2. The adduct according to claim 1, wherein the peroxide is hydrogen peroxide or urea peroxide.

3. The adduct according to claim 1, wherein the alkali metal pyrophosphate is selected from the group consisting of pyrophosphates of sodium or potassium, or any combinations thereof.

4. The adduct according to claim 1, wherein the alkali metal pyrophosphate is sodium pyrophosphate.

5. The adduct according to claim 1, wherein the polyamino acids comprise at least two amino acids selected from the group consisting of lysine, arginine, histidine, glycine, serine, threonine, asparagine, glutamine, cysteine, selenocysteine, proline, alanine, valine, isoleucine, leucine, methionine, phenylalanine, tyrosine, tryptophan, aspartic acid, glutamic acid or salts thereof, and any combinations thereof.

6. The adduct according to claim 5, wherein the polyamino acids comprise amino acids in the range of from about 2 to about 10,000.

7. The adduct according to claim 6, wherein the amino acids vary in the range of from about 20 to 100, or from about 100 to 1000, or from about 1000 to 10,000.

8. The adduct according to claim 1, wherein the proteins comprise from about 50 to about 50,000 amino acids selected from the group consisting of lysine, arginine, histidine, glycine, serine, threonine, asparagine, glutamine, cysteine, selenocysteine, proline, alanine, valine, isoleucine, leucine, methionine, phenylalanine, tyrosine, tryptophan, aspartic acid, glutamic acid, or salts thereof, and any combinations thereof.

9. The adduct according to claim 1, wherein the polyamino acid is polyaspartic acid or polyhistidine, or salts thereof.

10. The adduct according to claim 1, wherein the adduct comprises (i) from about 1.0 wt. % to about 50.0 wt. % of peroxide; (ii) from about 0.1 wt. % to about 99.0 wt. % of alkali metal pyrophosphate; and (iii) from about 0.1 wt. % to about 99.0 wt. % of at least one compound selected from the group consisting of polyamino acids, proteins, and any combinations thereof.

11. The adduct according to claim 1, wherein the adduct has a pH of about 4 to about 10.

12. A composition comprising the adduct of claim 1.

13. The composition according to claim 12, is a nutrition composition, a food composition, a beverage composition, a pharmaceutical composition, a laundry or cleaning composition, a coating composition, a bio-cide composition, a construction composition, an energy composition, an industrial composition, an oilfield composition, an oral care composition, a skin care composition, an acne treatment composition, a hair care composition, a scalp care composition, a household composition, a performance composition, an agricultural composition, a cosmetic composition, a medical device, a pesticide composition, a veterinary composition, a fuel composition, a lubricant composition, an adhesive composition, a textile composition, an ink composition, a membrane composition, or a disinfectant composition.

14. An oral care composition comprising:
(i) from about 0.5 wt. % to about 95.0 wt. % adduct of claim 1;
(ii) from about 10.0 wt. % to about 80.0 wt. % one or more orally acceptable excipients; and
(iii) from about 0.1 wt. % to about 20.0 wt. % one or more oral care active ingredients.

15. The oral care composition according to claim 14, wherein the one or more orally acceptable excipients are selected from the group consisting of thickening agents, desensitizing agents, whitening agents, tartar control agents, abrasives, binders, detergents, adhesion agents, foam modulators, pH modifying agents, mouth feel agents, sweeteners, flavorants, colorants, preservatives, humectants, fluoride containing salts and compounds, water, polymers, orally acceptable surfactants, stain preventors, encapsulants, and mixtures thereof.

16. The oral care composition according to claim 14, wherein the one or more oral care active ingredients are selected from the group consisting of an analgesic, an antibacterial, an antibiotic, a probiotic, an antioxidant, a peptide, an enzyme, a cooling agent, vitamins, and mixtures thereof.

17. The oral care composition according to claim 14, wherein the composition is formulated as a toothpaste, tooth gel, subgingival gel, tooth powder, mouth-rinse, mouth wash, denture cream, denture powder, mouth-spray, chewable tablet, dissolvable film, strip or chewing gum.

18. The oral care composition according to claim 14, wherein the composition is a non-aqueous composition.

19. The oral care composition according to claim 14, wherein the composition comprises water content in the range of from about 0.0% w/w to about 5.0% w/w.

20. The oral care composition according to claim 14, wherein composition has a pH in the range of from about 4 to about 10.

21. The oral care composition according to claim 14, wherein the adduct is present in an amount of from about 0.5 wt. % to about 25.0 wt. %, or from about 25.0 wt. % to about 50.0 wt. %, or from about 50.0 wt. % to about 75.0 wt. %, or from about 75.0 wt. % to about 95.0 wt. %, based on the total weight of the oral care composition.

22. An oral care composition comprising:
(i) from about 0.5 wt. % to about 95.0 wt. % adduct of claim 1 comprising (i) hydrogen peroxide; (ii) sodium pyrophosphate; and (iii) polyaspartic acid or polyhistidine, or salts thereof; and
(ii) from about 10.0 wt. % to about 80.0 wt. % one or more orally acceptable excipients; and
(iii) from about 0.1 wt. % to about 20.0 wt. % one or more oral care active ingredients.

23. The oral care composition according to claim 22, wherein the adduct is present in an amount of from about 0.5 wt. % to about 25.0 wt. %, or from about 25.0 wt. % to about 50.0 wt. %, or from about 50.0 wt. % to about 75.0 wt. %, or from about 75.0 wt. % to about 95.0 wt. %, based on the total weight of the oral care composition.

24. A process for preparing an adduct comprising the steps of:
(a) dispersing or dissolving about 0.1 wt. % to 99.0 wt. % of at least one alkali metal pyrophosphate and 0.1 wt. % to 99.0 wt. % of at least one compound selected from the group consisting of polyamino acids, proteins, and any combinations thereof in an aqueous peroxide solution at a temperature in the range of from about 4° C. to about 90° C. to obtain a solution or a slurry;
(b) stirring the resultant solution or slurry of step (a) from 1.0 rpm to 10,000 rpm and at a temperature of about 4° C. to about 90° C.;
(c) drying the resultant solution or slurry of step (b) at cold, room or higher temperature to form a dried adduct; and
(d) milling the dried adduct obtained in step (c) to obtain adduct with the desired particles size distribution

25. The process according to claim 24, wherein the drying is carried out by fluidized bed spraying, or spray drying, or belt or drum drying or wet and dry granulation, or agglomeration, or dry and wet compacting methods, or any combinations thereof.

26. A method to improve oral health comprising applying an effective amount of the oral composition of claim 14 to an oral cavity surface, wherein the method is effective (i) to reduce or inhibit formation of dental caries, (ii) for preventing the demineralization of the teeth, (iii) to promote remineralization of the teeth, (iv) to reduce or inhibit hypersensitivity of the teeth, (v) to treat gingivitis, erosion, cavities, calculus, inflammation, staining and plaque accumulation, and (vi) to reduce biofilm formation, (vii) enhance antibacterial activity, (viii) tooth whitening, and (xi) improve flavor retention.

27. A method to improve oral health according to claim 26, wherein the method is effective at delivering and retaining therapeutic agents including (i) antibacterial, (ii) anti-caries, (iii) anti-gingivitis, (iv) anti-inflammation, (v) anti-biofilm, (vi) anti-staining, and (vii) anti-erosion.

28. A method for whitening teeth comprising administering an oral care composition of claim 14 to the teeth surface.
